(19) United States
(12) Patent Application Publication
(10) Pub. No.: US 2026/0102630 A1
(43) Pub. Date: Apr. 16, 2026

(54) Light Stimulation Apparatus

(71) Applicant: Aleddra Inc., Renton, WA (US)

(72) Inventors: Li-Jyuan Luo, Taipei City (TW); Chia-Yiu Maa, Bellevue, WA (US)

(73) Assignee: Aleddra Inc., Renton, WA (US)

(21) Appl. No.: 19/368,799

(22) Filed: Oct. 24, 2025

Related U.S. Application Data
(63) Continuation-in-part of application No. 19/186,404, filed on Apr. 22, 2025.
Application No. 19/186,404 is a continuation-in-part of application No. 18/626,148, filed on Apr. 3, 2024.
Application No. 18/626,148 is a continuation-in-part of application No. 18/613,079, filed on Mar. 21, 2024.
Application No. 18/613,079 is a continuation-in-part of application No. 18/408,523, filed on Jan. 9, 2024, now Pat. No. 12,507,332.

(51) Int. Cl. A61N 5/06; A61M 21/02; A61M 21/00

(52) U.S. Cl. CPC A61N 5/0622; A61M 21/02; A61M 2021/0027; A61N 2005/0648; A61N 2005/0653; A61N 2005/0659; A61N 2005/0663

(57) ABSTRACT

A lighting apparatus includes a light source and a controller. The light source emits a red light in a range of 600-700nm or a near-infrared (NIR) light in a range of 760-950nm. The controller is configured to generate an electrical pulse signal with a pulse frequency between 3 and 10Hz and to operate the light source according to the pulse frequency. The lighting apparatus is suitable for applications regulating cytochrome c oxidase (CcO), modulating transient receptor potential (TRP) channels, and affecting mitochondrial function, thereby potentially contributing to the reduction of amyloid-beta protein accumulation and improvement of cognitive function of a subject.

### BACKGROUND

The present disclosure is a continuation-in-part (CIP) of U.S. patent application Ser. No. 19/186,404, filed 22 April 2025, which is a CIP of US Patent Application No. 18/626,148 filed 3 April 2024. Content of aforementioned applications are herein incorporated by reference in their entirety.

### TECHNICAL FIELD

The present disclosure pertains to the field of lighting apparatus, and more specifically, proposes lighting apparatus with theta stimulation using red or near infrared (NIR) light.

### DESCRIPTION OF RELATED ART

In U.S. patent application Ser. No. 19/186,404, a wearable light device with dual frequencies was introduced to strobe with two flicker-free lights at a frequency equal to the difference of the dual frequencies. Such frequency is induced endogenously in the brain of a subject. Some embodiments further included NIR LED emitting 850 nm wavelength, for additional health benefits such as improving blood circulation, lowering heartbeat, and improving the mood of the subject.

While the invention addressed broad NIR exposure for wellness, some other studies have reported that specific combinations of wavelength and pulsing frequency can modulate mitochondrial dynamics and neuronal activity, potentially contributing to better therapeutic outcomes in treating Alzheimer’s disease.

Recently, it has been discovered that exposing Alzheimer’s disease mouse model to 40Hz pulsed light at a wavelength of approximately 808nm was associated with improved spatial learning and memory abilities and more efficient phototherapy. The underlying mechanism is believed to involve absorption of light (650nm – 950nm) by mitochondrial cytochrome c oxidase (CcO). This metabolic modulation increases the population of anti-inflammatory microglia, enhances Aβ phagocytosis, and reduces excessive reactive oxygen species (ROS) generation, thereby contributing to neuronal preservation. (Xu et. al., Journal of Photochemistry and Photobiology B: Biology (2024) 250:112816. https://doi.org/10.1016/j.jphotobiol.2023.112816).

Another study has shown that exposing 10Hz pulsed light was more effective as compared to 40Hz pulsed light at a wavelength of approximately 1070nm. One explanation is that the 100ms period is similar to the activation dynamics of transient receptor potential (TRP) channels and/or calcium ion channels that can respond to light stimulation. A pulsed modulation may activate TRP channels, leading to specific physiological or neuronal responses associated with visual or cognitive modulation. (Tao et al., Light: Science & Applications (2021) 10:179. https://doi.org/10.1038/s41377-021-00617-3).

TRP channel-mediated depolarization can be achieved from synaptic activation at low frequencies of 1Hz – 10Hz. Another study has found that, in humans, “slow theta” (3Hz – 4Hz) stimulation is predictive of correct recall and corresponds functionally to the 7Hz theta stimulation of rats. (Lisman et. al, (2013) 77:1002-1016. https://doi.org/10.1016/j.neuron.2013.03.007). A similar study has confirmed that the theta phase of simultaneously presented auditory and visual stimuli can be used to influence or enhance associative memory formation in the subject. (Köster et. al, Neuroimage (2019) 188:181-187. https://doi.org/10.1016/j.neuroimage.2018.12.002).

The advancement of recent research reports cited above leads to the idea of augmenting the lighting apparatus described in U.S. patent application Ser. No. 19/186,404 with a light source operating at a pulse frequency between 3 and 10 Hz for treating or preventing Alzheimer’s disease.

### SUMMARY

In one aspect, the lighting apparatus comprises a light source emitting a light in a wavelength range from 600nm to 950nm and a controller, The controller is configured to generate an electrical pulse signal with a pulse frequency between 3Hz and 10Hz and operate the light source according to the pulse frequency. Unlike U.S. patent application Ser. No. 19/186,404 where two visible light sources with dual frequencies are used to induce endogenously gamma stimulation in the brain of the subject, the present disclosure relies on one single light source for trigger theta stimulation to the subject through completely different path in the body of the subject, resulting a similar benefit of treating or preventing Alzheimer’s disease.

In order to specifically target CcO for modulating the mitochondrial activity of the subject, in some embodiments, the light source emits the light at wavelength(s) aiming at main absorption peak of mitochondrial CcO. One such wavelength is 655nm corresponding to the fully oxidized haem α3/CuB binuclear center. Another such wavelength is 835nm corresponding to CuA center.

In some embodiments, the light source comprises a light emitting diode (LED) or organic LED (OLED). LED and OLED light source can be turned on/off at a high frequency and at different intensity easily, thus suitable for supporting strobing operation in some embodiments.

In some embodiments, the light pulsing at the pulse frequency between 3Hz and 10Hz has the effect of activating transient receptor potential (TRP) channels to modulate calcium dynamics and affect mitochondrial function in the brain or other neural tissues of the subject。

In some embodiments, the power density of the light emitted by the light source to the subject is between 1 and 10 mW/cm². This ensures the light is bright enough to create sufficient stimulation to the subject.

In some embodiments, the controller is configured to operate the light source in a continuous wave (CW) mode or a pulsed wave (PW) mode. The CW may be preferred for it leads to a better stimulation of the subject’s brain activity.

In some embodiments, the light stimulation apparatus is embodied as a wearable device with a wearing mechanism. The wearable device is likely powered by an internal battery, or it may be powered by an external power source. There is various wearing mechanisms demonstrated by different electronic goggles on the market. Some use an elastic strap to fasten the goggles on the head of the subject. Some others take the shape of an eyeglass with a bridge fitting over the nose of the subject and two temples over the ears of the subject. Some others may use a wire frame to fit over the head of the subject. It is to be noted the wearing mechanism is not limited to the wearing mechanism over the head of the subject.

In some embodiments, the light stimulation apparatus is embodied as a portable device. The portable device may be powered by an internal battery, a replaceable battery pack, or an external power supply by using a USB or wireless charging system. In some embodiments, the portable device is designed for flexible use in various environments without requiring fixed installation. Some others may be used in clinical, hospital, home and can be operated independently of a stationary power source. Some others allow flexible use of the apparatus in clinical, home, or mobile environments without requiring the user to wear the device.

In some embodiments, the light stimulation apparatus further includes a sound generator. The sound generator generates an audible sound at the same pulse frequency between 3Hz and 10Hz. The sound generator may be a standalone device or a component of the controller. In some other embodiments, the sound generator generates an audible sound at 40Hz.

Further in some embodiments, the sound generator generates a sinusoidal sound wave, as opposed to a square sound wave. A square sound wave is harsher and more uncomfortable for ears as compared to a sinusoidal sound wave. Thus, using a sinusoidal sound wave may lead to better user adoption or longer treatment time, resulting more effective treatment of Alzheimer's disease.

In some embodiments, the light stimulation apparatus further includes an optical filter disposed adjacent to the light source for the light source for shifting and/or centering the wavelength of the light around 655nm or 835nm. The optical filter enables the use of commercially available light sources (e.g., 660nm or 850nm LEDs) while effectively achieving the optimal targeting of the CuA and haem α3/CuB binuclear center.

### BRIEF DESCRIPTION OF THE DRAWINGS

The accompanying drawings are included to aid further understanding of the present disclosure and are incorporated in and constitute a part of the present disclosure. The drawings illustrate a select number of embodiments of the present disclosure and, together with the detailed description below, serve to explain the principles of the present disclosure. It is appreciable that the drawings are not necessarily to scale, as some components may be shown to be out of proportion to size in actual implementation in order to clearly illustrate the concept of the present disclosure.

FIG. 1, schematically depicts a first embodiment of the present disclosure with a light source and a controller configured to generate an electrical pulse signal with a pulse frequency between 3Hz and 10Hz.

FIG. 2, schematically depicts a second embodiment of the present disclosure with a light source, a sound generator, and a controller configured to generate an electrical pulse signal with a pulse frequency between 3Hz and 10Hz.

FIG. 3, schematically depicts a third embodiment of the present disclosure with a light source, an optical filter, and a controller configured to generate an electrical pulse signal with a pulse frequency between 3Hz and 10Hz.

### DETAILED DESCRIPTION OF PREFERRED EMBODIMENTS

### Overview

Various implementations of the present disclosure and related inventive concepts are described below. It should be acknowledged, however, that the present disclosure is not limited to any particular manner of implementation, and that the various embodiments discussed explicitly herein are primarily for purposes of illustration. For example, the various concepts discussed herein may be suitably implemented in a variety of light stimulation apparatus having different form factors.

A light stimulation apparatus comprises at least one light source and controller. The light source configured to emit a light in a wavelength range from 600nm to 950nm, and the controller configured to operate the light pulsing at a pulse frequency between 3Hz and 10Hz. This apparatus is used for modulating mitochondrial CcO activity and transient receptor potential (TRP) channel function of a subject.

### Example Implementations

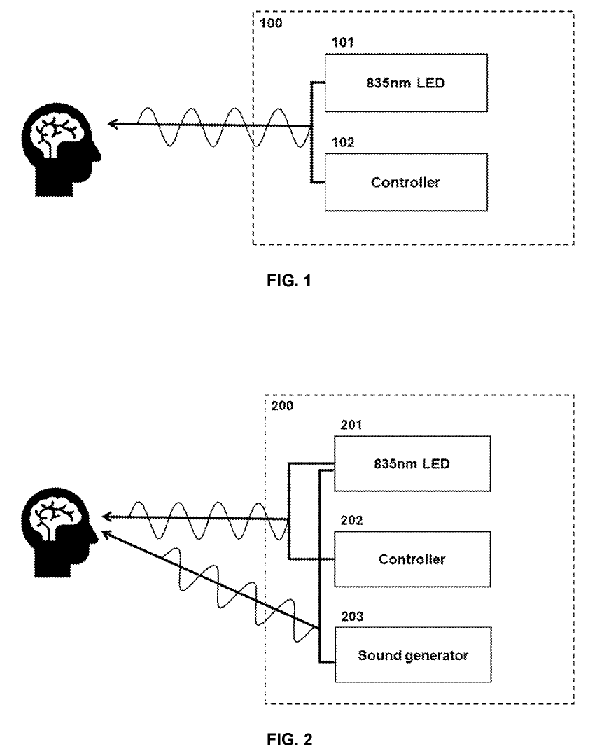

FIG. 1 is the first embodiment of the light stimulation apparatus of the present disclosure. The embodiment 100 has a light source 101 and a controller 102. The light source 101 emits light at 835nm and the controller 102generates an electrical pulse signal with a pulse frequency between 3Hz and 10Hz and operates the light source 101 according to the pulse frequency.

Given that 835 nm wavelength is invisible, a user of the first embodiment 100 will not see any flickering between 3Hz and 10Hz, thus getting the benefit of targeting to the fully oxidized haem α3/CuB binuclear center for treating or preventing Alzheimer’s disease.

One variation of embodiment 100 is to replace light source 101 to 655 nm LED, corresponding to the fully oxidized haem α3/CuB binuclear center. However, when using the 655 nm LED, attention should be paid to the flickering effect at 3 – 10 Hz, since the 655 nm wavelength is within the visible spectrum.

In another variation of embodiment 100, the controller is configured to generate an electrical pulse signal between 3Hz and 4Hz (narrower than 3Hz to 10Hz), which has been found to be predictive of correct recall.

FIG. 2 is the second embodiment of the light stimulation apparatus of the present disclosure. The embodiment 200 has a light source 201, a controller 202, and a sound generator 203. The light source 201 emits light at 835nm and the controller 202 generates an electrical pulse signal with a pulse frequency between 3Hz and 10Hz. The sound generator generates a sinusoidal sound wave which may lead to better user adoption or longer treatment time, resulting a more effective treatment of Alzheimer's disease.

In one variation of embodiment 200, the sound generator 203 is configured to generate a sinusoidal sound having a pulse frequency at 40 Hz. This embodiment presents simultaneously auditory at 40Hz and visual stimuli at 3 – 10Hz to influence or enhance associative memory formation.

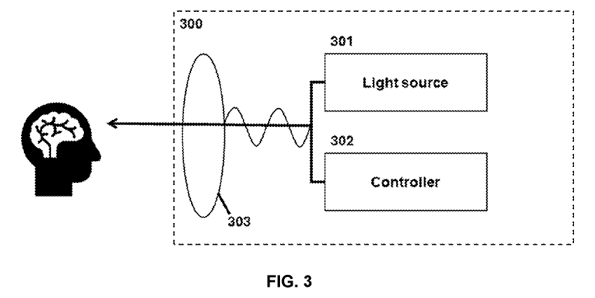

FIG. 3 is the third embodiment of the light stimulation apparatus of the present disclosure. The embodiment 300 has a light source 301, a controller 302, and an optical filter 303. The optical filter 303 disposed adjacent to the light source for the light source for shifting and/or centering the wavelength of the light around 655nm or 835nm. The optical filter enables the use of commercially available light sources (e.g., 660nm or 850nm LEDs) while effectively achieving the optimal targeting of the CuA and haem α3/CuB binuclear center.

This filter technology allows an arbitrary transmission characteristic. This filter is advantageously located over or in front of the entire light stimulation apparatus area.

### Additional and Alternative Implementation Notes

Although the techniques have been described in language specific to certain applications, it is to be understood that the appended claims are not necessarily limited to the specific features or applications described herein. Rather, the specific features and examples are disclosed as non-limiting exemplary forms of implementing such techniques.

As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more,” unless specified otherwise or clear from context to be directed to a singular form.

What is claimed is:

1. A light stimulation apparatus, comprising:
a light source configured to emit a light in a wavelength range from 600 to 950nm; and
a controller configured to generate an electrical pulse signal with a pulse frequency between 3Hz and 10Hz and operate the light source according to the pulse frequency.

2. The light stimulation apparatus of claim 1, wherein the light source emits the light in a wavelength range from 600 to 700nm.

3. The light stimulation apparatus of claim 2, wherein the light at 655nm triggers an absorption peak of cytochrome c oxidase (CcO) in a brain or other neural tissues of a subject.

4. The light stimulation apparatus of claim 1, wherein the light source emits the light in a wavelength range from 760 to 950nm.

5. The light stimulation apparatus of claim 4, wherein the light at 835nm triggers an absorption peak of CcO in a brain or other neural tissues of a subject.

6. The light stimulation apparatus of claim 1, wherein the light source comprises a light emitting diode (LED) or organic LED (OLED).

7. The light stimulation apparatus of claim 1, wherein the light pulsing at the pulse frequency between 3Hz and 10Hz has an effect of activating transient receptor potential (TRP) channels to modulate calcium dynamics and affect mitochondrial function in a brain or other neural tissues of a subject.

8. The light stimulation apparatus of claim 1, wherein a power density of the light emitted by the light source to a subject is between 1 and 10 mW/cm².

9. The light stimulation apparatus of claim 1, wherein the controller is configured to operate the light source in either of a continuous wave (CW) mode and a pulsed wave (PW) mode.

10. The light simulation apparatus of claim 1, wherein the apparatus has a form factor of a wearable device with a wearing mechanism.

11. The light simulation apparatus of claim 1, wherein the apparatus has a form factor of a portable device.

12. The light stimulation apparatus of claim 1, further comprising a sound generator configured to generate an audible sound at the pulse frequency.

13. The light stimulation apparatus of claim 12, wherein the sound generator is configured to generate a sinusoidal sound wave.

14. The light stimulation apparatus of claim 1, further comprising a sound generator configured to generate an audible sound at 40Hz.

15. The light stimulation apparatus of claim 14, wherein the sound generator is configured to generate a sinusoidal sound wave.

16. The light stimulation apparatus of claim 1, further comprising an optical filter disposed adjacent to the light source and configured to either or both shift and center a wavelength of the light around 655nm or 835nm.
